(19) United States
(12) Patent Application Publication
(10) Pub. No.: US 2011/0236859 A1
(43) Pub. Date: Sep. 29, 2011

(54) ORAL CARE COMPOSITION CONTAINING OXIDASE ENZYME

(76) Inventors: Bradley R. Keleman, Menlo Park, CA (US); Erin Wolger, San Mateo, CA (US)

(21) Appl. No.: 12/989,302

(22) PCT Filed: Mar. 30, 2009
(86) PCT No.: PCT/US09/38777

§ 371 Date: Jun. 3, 2011

(51) Int. Cl. A61K 8/84; A61P 31/04; A61Q 11/00; A61C 17/00; A61C 15/04

(52) U.S. Cl. 433/216; 424/50

(57) ABSTRACT

Oral care compositions comprising an oxidase, polyethyleneimine, and an orally acceptable carrier are described, along with methods of use thereof. The oral care composition may be applied to a tooth surface to produce peroxide.

### PRIORITY

The present application claims priority to U.S. Provisional Patent Application Ser. No. 61/047,011, filed on Apr. 22, 2008, which is herein incorporated by reference in its entirety.

### TECHNICAL FIELD

The compositions and methods are in the field of oral care, and involve the to application of composition comprising an oxidase and polyethyleneimine to a tooth surface to produce peroxide.

### BACKGROUND

Human teeth are constructed with an inner soft layer called dentin and an outer hard layer called enamel that protects the dentin. The enamel layer of teeth is translucent white or slightly off-white in color. Under typical conditions, the enamel surface of a tooth is coated with a thin layer of salivary proteins called pellicle. The enamel, the pellicle, or both can be become discolored or stained, presenting an undesirable appearance. Further, the enamel layer of teeth is believed to be porous in nature allowing staining components and discoloring substances to penetrate into the tooth structure, making discoloration and staining difficult to remove.

In addition to cosmetic issues, good oral hygiene is important for maintaining oral cavity health. Effective oral hygiene requires the control of dental plaque. Dental plaque is a mixed matrix of bacteria, epithelial cells, leukocytes, macrophages and other oral exudate. The formation of dental plaque is the primary source of dental caries, gingivitis, periodontal disease, and tooth loss.

The need exists for compositions and methods to improve oral hygiene for both cosmetic and health reasons.

### SUMMARY

Described are an oral care composition and method involving polyethyleneimine and an uncomplexed oxidase. The composition may include an orally acceptable carrier. In certain cases, the oxidase may be a glucose oxidase, although other oxidases may be used. In particular embodiments, the polyethyleneimine and the uncomplexed glucose oxidase are present in amounts sufficient for binding of the oxidase to an enamel surface of a tooth via the polyethlyneimine and production of peroxide in the presence of a suitable substrate for the oxidase.

The polyethyleneimine may present in said oral care composition at a concentration in the range of 0.001% to 10% by weight, and the oxidase may be present in the oral care composition at a concentration in the range of 0.0001% to 1% by weight.

The oral care composition may contain at least one of a thickener, a surfactant, a to humectant, and an abrasive and, in certain cases, a second enzyme in addition to the oxidase. In certain cases, the oxidase in the composition is not complexed with another enzyme.

The oral care composition may be formulated as, for example, a fluid, e.g., a toothpaste, or as a solid, e.g., as a powder or tooth floss.

The oral care composition may be employed in a method that comprises: contacting the oral care composition with an enamel surface of a tooth under conditions suitable for binding of the oxidase to said enamel surface and production of peroxide. The method may be for producing peroxide on a tooth surface.

The contacting may be done using a toothbrush or tooth floss, for example. In particular cases, the method may result in death of bacteria on the tooth and/or tooth whitening.

Also provided is a method of binding oxidase to a tooth. In certain cases, this method may comprise: contacting a tooth having an enamel surface with an oxidase in the presence of polyethyleneimine or sorbitol under conditions that provide for binding of said oxidase to said enamel surface via said polyethyleneimine or said sorbitol and production of peroxide. The contacting may be performed using a toothbrush or tooth floss, for example.

These and other aspects and embodiments of the present oral care compositions and methods are described in more detail, below.

### BRIEF DESCRIPTION OF THE FIGURES

FIG. 1 shows a graph demonstrating the binding of glucose oxidase to hydroxylapatite in the presence of varying concentrations of sorbitol or polyethyleneimine. Glucose oxidase binding to saliva coated hydroxyappetite disks is measured using a fluorescent (Amplex Red) horseradish peroxidase coupled assay directly from the disk surface. The additives sorbitol (8, 0.8, 0.08% shown in individually labeled triangles), and polyethylenimine (1% shown in diamonds) improve binding relative to no additive (in squares).

### DETAILED DESCRIPTION

### I. Definitions

Unless defined otherwise, all technical and scientific terms should be accorded their ordinary meaning as used in the art. The following terms are defined below for the sake of clarity and ease of reference. Other definitions may appear throughout the specification.

As used herein, the term “oral care composition” refers to a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral composition of the present invention may be in the form of a toothpaste, dentifrice, tooth powder, tooth gel, subgingival gel, mouthrinse, denture product, mouthspray, lozenge, oral tablet, or chewing gum, for example. The oral composition may also be incorporated onto strips, films or floss for direct application or attachment to oral surfaces.

As used herein, the term “dentifrice” refers to paste, gel, solid or liquid formulations, unless otherwise specified. The dentifrice composition may be a single-phase composition or may be a combination of two or more separate dentifrice compositions. The dentifrice composition may be in any desired form, such as deep striped, surface striped, multilayered, having the gel surrounding the paste, or any combination thereof. Each dentifrice composition in a dentifrice comprising two or more separate dentifrice compositions may be contained in a physically separated compartment of a dispenser and dispensed together simultaneous or sequentially, e.g., in a side-by-side arrangement.

As used herein, the term “orally acceptable carrier” refers to any safe and effective material(s) for use in oral care compositions. Such materials include fluoride ion sources, anticalculus agents, buffers, abrasive polishing materials, peroxide sources, alkali metal bicarbonate salts, thickening materials, humectants, water, surfactants, titanium dioxide, flavor system, sweetening agents, xylitol, coloring agents, and mixtures thereof.

As used herein, the term “tooth” or “teeth” encompasses natural teeth as well as artificial teeth or dental prosthesis.

As used herein, the term “enamel” refers to the part of a tooth that is normally visible and is composed of mostly minerals, including hydroxylapatite. Enamel encompasses naturally-occurring enamels in teeth of humans and animals as well as enamel-like substance used to replace damaged or missing teeth parts, including resins and porcelains used for such purposes.

As used herein, the terms “tartar” and “calculus” are used interchangeably to refer to mineralized dental plaque deposits.

As used herein, the term “polyethyleneimine,” abbreviated “PEI,” refers to a polymer made from ethyleneimines Polyethyleneimine may exist in a protonated or unprotonated form, with or without water. Polyethyleneimine encompasses branched and unbranched polyamines, as well as salts thereof, including PEI-hydrochloride salt, PEI-sulfuric acid salt, PEI-nitric acid salt, PEI-acetic acid salt, PEI-fatty acid salt, and the like. PEI has the empirical formula (C₂H₅N)ₙ with each repeating unit having a molecular weight of 43.07.

As used herein, the term “complexed” describes an enzyme (which, itself may be a multimer, e.g., a dimer containing two subunits) that is directly or indirectly linked to another protein via a covalent or non-covalent bond.

As used herein, the term “uncomplexed” describes an enzyme (which, itself may be a multimer, e.g., a dimer containing two subunits or a trimer) that is not linked to another protein, directly or indirectly, via a covalent or non-covalent bond. For example, “uncomplexed glucose oxidase” refers to a glucose oxidase (which may be a dimer or trimer) that is not conjugated or linked to another enzyme or antibody.

As used herein, the term “substantially active,” with reference to the present enzyme compositions, means that the enzyme(s) has an activity that is at least 30%, at least 50%, at least 70%, or at least 80% of its optimum or maximum activity.

As used herein “compatible,” means that the components, e.g., of a composition, are capable of being commingled without interacting in a manner that would substantially reduce the stability and/or efficacy of the composition.

As used herein, the term “lozenge” includes but is not limited to: breath mints, troches, pastilles, microcapsules, and fast-dissolving solid forms including freeze dried forms (cakes, wafers, thin films, tablets) and compressed tablets.

As used herein, the term “fast-dissolving solid form” means that a solid dosage form dissolves in less than about 60 seconds, less than about 15 seconds, or less than about 5 seconds, e.g., after placing the solid dosage form in the oral cavity or a container to containing dental prosthetics.

As used herein, a “subject,” “consumer,” or “end-user” is the individual whose tooth surface (including the surface or dental implants) contacts the oral composition. The individual may be a human or an animal Animals include household pets or other domestic animals, or animals kept in captivity.

Numeric ranges are inclusive of the numbers defining the range. All percentages and ratios used herein are by weight of the specific oral composition and not of the overall oral formulation that is delivered, unless otherwise specified.

Headings are provided for ease of reading but should not be construed as limitations, as the contents under different headings may apply to the description as a whole. The terms “a,” “an,” and “the,” include the plural unless otherwise apparent from context.

Although any methods and materials similar or equivalent to those described herein can be used in the practice or testing of the present invention, the preferred methods and materials are described.

All patents and publications, including all sequences disclosed within such patents and publications, referred to herein are expressly incorporated by reference.

### II. Oral Care Compositions

An oral care composition comprising polyethyleneimine and an uncomplexed oxidase is provided. Without being limited to a theory, it is believed that polyethyleneimine promotes the adhesion of the oxidase to hydroxyappetite on the surface of teeth, thereby maximizing the effect of the oxidase in terms of providing a cleaning benefit. The components of the composition and exemplary methods of their use are further described, below.

### A. Oxidase

The oxidase may be any oxidase capable of generating peroxide (H₂O₂) in situ (i.e., in the oral cavity) and that has antimicrobial and/or tooth whitening/cleaning activity. Exemplary oxidases include, but are not limited to, glucose oxidase, hexose oxidase, L-amino-acid oxidase, xylitol oxidase, galactose oxidase, pyranose oxidase, and alcohol oxidase. The oxidase may be obtained from a suitable organism or produced from a recombinant source. The glucose oxidase may be a recombinant version of a wild type enzyme or a variant. The amino acid sequences of many oxidases are known and are deposited in NCBI's Genbank database, and can be used to select an oxidase for expression and isolation. Suitable glucose oxidases may originate from various organisms, including an Aspergillus spp., such as a strain of Aspergillus niger, or a Cladosporium spp., such as Cladosporium oxysporum. Oxidases may also be purchased from commercial suppliers, e.g., AppliChem GmbH (Darmstadt, Germany), Sigma-Aldrich (St. Louis, Mo., USA) USB Corp., (Cleveland, Ohio, USA), and others.

In a particular embodiment, the oxidase is a glucose oxidase (EC1.1.3.4/CAS number 9001-37-0). This enzyme binds to beta-D-glucose (an isomer of the six-carbon sugar glucose) and aids in breaking it down. In certain cases, glucose oxidase may be a dimeric protein that catalyzes the oxidation of beta-D-glucose into D-glucono-1,5-lactone, which then hydrolyzes to gluconic acid. The glucose oxidase may employ a cofactor such as flavin adenine dinucleotide (FAD). In a glocose-oxidase catalyzed redox reaction, FAD acts as the initial electron acceptor and is reduced to FADH₂, which is subsequently oxidized by the final electron acceptor, molecular oxygen (O₂). O₂ is then reduced to produce hydrogen peroxide (H₂O₂).

In another embodiment, the oxidase is a hexose oxidase (EC 1.1.3.5), which catalyzes a reaction between D-glucose and O₂ to produce D-glucono-1,5-lactone and H₂O₂. The hexose oxidases may be from the red sea-weed Chondrus crispus (commonly known as Irish moss) which oxidises a broad spectrum of carbohydrates, such as D-glucose, D-galactose, maltose, cellobiose, lactose, D-glucose 6-phasphate, D-mannose, 2-deoxy-D-glucole, 2-deoxy-D-galactose, D-fucase, D-glucurnic acid, and D-xylose. The red sea-weed Iridophrycus flaccidum hexose oxidases, which oxidize several different mono- and disaccharides, may also be used. These oxidases may be employed in conjunction with a copper cofactor.

In another embodiment, the oxidase is a xylose oxidase (EC 1.1.3.41), which is an enzyme that catalyzes a reaction between xylitol and O₂ to produce xylose and H₂O₂. Certain xylitol oxidases (see, e.g., JP 80892242) oxidizes xylitol, D-sorbitol, D-galactitol, D-mannitol and D-arabinitol in the presence of oxygen. A xylitol oxidase can be obtained, for example, from strains of Streptomyces spp. These enzymes have a pH optimum of 7.5 and are stable at pH 5.5 to 10.5 and at temperatures up to 65° C.

In particular embodiments, the oxidase may be present in a composition at a concentration in the range of about 0.0001% to about 1%, e.g., 0.001% to 0.1%.

### B. Polyethyleneimine

Polyethyleneimine (CAS 9002-98-6, 26913-06-4) is a cationic polymer that binds to certain proteins. It is used to precipitate and purify enzymes and lipids and as a marker in immunology. Polyethyleneimine is also known as aziridine polymer, epamine, epomine, ethylenimine polymer, and PEI, and is available from commercial chemical supply companies.

In the present compositions, polyethyleneimine may be present at a concentration in the range of about 0.01% to 10%, e.g., 0.1% to 5%.

### C. Carriers and Formulations

The present compositions may include an orally acceptable carrier. Generally, the present oral care compositions are formulated so that the oxidase enzyme is stable in an active or inactive form, and remains or becomes active when the oral care composition is contacted with the enamel surface of a tooth. In some embodiments, the enzyme is active on a tooth surface after the composition has been contacted with the tooth, and the remainder of the composition rinsed away, e.g., in the case of using a toothpaste formulation, and then rinsing. Thus, the oxidase may substantially active under pH conditions that prevail in the mouth, i.e., between pH 5.0 to 9.0, between pH 6.0 to 8.5, or even between pH 6.4 to 7.5, or in the oral care composition.

In some embodiments, the oral care composition contains a substrate for the oxidase, e.g., a sugar or alcohol, and/or a cofactor necessary for the activity of the oxidase, e.g., FAD, copper, and the like. In other embodiments, the substrate for the oxidase may be provided in a composition, e.g., a foodstuff, that is not the oral care composition. In one embodiment, the oxidase may be applied to the enamel surface of a tooth and linked thereto, and a foodstuff containing a substrate for the enzyme (e.g., a sugar-containing food such as a candy, pastry or drink) may be contacted with the tooth at a later time. Suitable substrates depend on the oxidase used. For example, if the oxidase is a glucose oxidase, glucose may be used as a substrate and if the oxidase is hexose oxidase, then hexose may be used as a substrate. If a subject oral care composition contains a substrate, it may be present at a concentration in the range of 0.1% to 5%, e.g., 0.5% to 2%, by weight, although concentrations outside of these ranges are envisioned.

The oral care composition may be in the form of a dentifrice, toothpaste, tooth powder, topical oral gel, mouthrinse, denture product, mouthspray, lozenge, oral tablet, or chewing gum, for example. The oral composition may also be incorporated onto strips, films or floss for direct application or attachment to oral surfaces.

An orally acceptable carrier may comprise one or more compatible solid or liquid filler diluents or encapsulating substances, which are suitable for topical oral administration. The carriers or excipients of the composition may include the usual and conventional components of dentifrices (including non-abrasive gels and gels for subgingival application), mouth rinses, mouth sprays, chewing gums, and lozenges (including breath mints) as more fully described hereinafter. In aqueous form, the oral care compositions may have a pH ranging from about 4.0 to about 10.0, e.g., from about 5.0 to about 8.0.

In certain embodiments, the carrier is selected based on the way the oral care composition is to be introduced into the oral cavity. For example, if a toothpaste (including tooth gels, etc.) is intended, then a “toothpaste carrier” may be selected, which comprises e.g., abrasive materials, surfactants, binders, humectants, flavoring and sweetening agents, etc., as described in e.g., U.S. Pat. No. 3,988,433, to Benedict. If a mouth rinse is intended, then a “mouthrinse carrier” may be selected, which comprises e.g., water, flavoring and sweetening agents, etc., as described in the same reference. Similarly, if a mouth spray is intended, then a “mouth spray carrier” may be selected, or if a lozenge is intended, then a “lozenge carrier” may be selected (e.g., a candy base). If a chewing gum is intended, a “chewing gum carrier” may be selected (comprising e.g., gum base, flavoring and sweetening agents). If a sachet is intended, then a “sachet carrier” may be selected (e.g., sachet bag, flavoring and sweetening agents). If a subgingival gel is intended (e.g., for delivery of actives into the periodontal pockets or around the periodontal pockets), then a “subgingival gel carrier” may be selected. Other useful carriers suitable for the preparation of the present compositions are well known in the art. The selection of carriers may depend on secondary considerations, such as taste, cost, shelf stability, and the like.

In some embodiments, the composition may be in the form of a non-abrasive gel, to e.g., a subgingival gel, which may be aqueous or non-aqueous. Aqueous gels generally include a thickening agent (from about 0.1% to about 20%), a humectant (from about 10% to about 55%), a flavoring agent (from about 0.04% to about 2%), a sweetening agent (from about 0.1% to about 3%), a coloring agent (from about 0.01% to about 0.5%), and the balance water. The composition may comprise an anticaries agent (from about 0.05% to about 0.3% as fluoride ion), and an anticalculus agent (from about 0.1% to about 13%).

In other embodiments, the composition may be in the form of a dentifrice, such as a toothpaste, tooth gel or tooth powder. Components of such toothpaste and tooth gels may include one or more of a dental abrasive (from about 5% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an anticaries agent (from about 0.05% to about 0.3% as fluoride ion), and an anticalculus agent (from about 0.1% to about 13%). Tooth powder may contain substantially all non-liquid components.

In some embodiments, the composition is a mouthwash, including a mouth spray. Components of mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Mouthwashes and mouth sprays may also include one or more of an anticaries agent (from about 0.05% to about 0.3% as fluoride ion), and an antic alculus agent (from about 0.1% to about 3%).

In some embodiments, the composition is a dental solution including an irrigation fluid. Components of dental solutions generally include one or more of water (from about 90% to about 99%), preservative (from about 0.01% to about 0.5%), thickening agent (from 0% to about 5%), flavoring agent (from about 0.04% to about 2%), sweetening agent (from about 0.1% to about 3%), and surfactant (from 0% to about 5%).

In some embodiments, the composition is a chewing gum. Chewing gum compositions typically include one or more of a gum base (from about 50% to about 99%), a flavoring agent (from about 0.4% to about 2%) and a sweetening agent (from about 0.01% to about 20%).

In some embodiments, the composition is a lozenge. Lozenges may include discoid-shaped solids comprising a therapeutic agent in a flavored base. The base may be a hard sugar candy, glycerinated gelatin or combination of sugar with sufficient mucilage to give it form. These dosage forms are generally well known in the art.

In some embodiment, the composition is in the form of a dental implement impregnated with the oral care composition. The dental implement may comprise an implement for contact with teeth and other tissues in the oral cavity, which implement is impregnated with a composition comprising an oxidase and polyethyleneimine. The dental implement may be impregnated fibers including dental floss or tape, chips, strips, films, toothpicks, and polymer fibers.

Exemplary materials that may be present in an orally acceptable carrier are described below.

1. Abrasives

Dental abrasives may include many different materials. The materials selected should be compatible within the other components in the oral care composition and should not excessively abrade dentin. Suitable abrasives include, for example, silicas such as gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

One class of abrasives for use in the compositions is a particulate thermo-setting polymerized resin. Suitable resins include, for example, melamines, phenolics, ureas, melamine-ureas, melamine-formaldehydes, urea-formaldehyde, melamine-urea-formaldehydes, cross-linked epoxides, and cross-linked polyesters.

Silica and other dental abrasives of various types may be selected because of their benefits dental cleaning and polishing performance without unduly abrading tooth enamel or dentine. The silica abrasive polishing materials, as well as other abrasives, may have an average particle size ranging between about 0.1 to about 30 microns, or from about 1 to about 15 microns. The abrasive can be precipitated silica or silica gels such as the silica to xerogels.

Mixtures of abrasives may be used. The total amount of abrasive in dentifrice compositions of the subject invention may range from about 6% to about 70% by weight. Toothpastes may contain from about 10% to about 50% of abrasives, by weight of the composition. Solution, mouth spray, mouthwash and non-abrasive gel compositions of may not include an abrasive.

2. Surfactants

The oral care composition may also contain a surfactant, e.g., a sarcosinate surfactant, isethionate surfactant or taurate surfactant. In certain embodiments, the composition may contain alkali metal or ammonium salts of these surfactants. The composition may contain sodium and potassium salts of the following: lauroyl sarcosinate, myristoyl sarcosinate, palmitoyl sarcosinate, stearoyl sarcosinate and oleoyl sarcosinate. Other suitable compatible surfactants may be used or in combination with a sarcosinate or other surfactant.

Anionic surfactants include the water-soluble salts of alkyl sulfates having from 10 to 18 carbon atoms in the alkyl radical and the water-soluble salts of sulfonated monoglycerides of fatty acids having from 10 to 18 carbon atoms. Sodium lauryl sulfate and sodium coconut monoglyceride sulfonates are examples of anionic surfactants of this type. Mixtures of anionic surfactants may also be utilized.

Cationic surfactants may be broadly defined as derivatives of aliphatic quaternary ammonium compounds having one long alkyl chain containing from about 8 to 18 carbon atoms such as lauryl trimethylammonium chloride; cetyl pyridinium chloride; cetyl trimethylammonium bromide; di-isobutylphenoxyethyl-dimethylbenzylammonium chloride; coconut alkyltrimethylammonium nitrite; cetyl pyridinium fluoride; etc. In some cases, surfactant compounds may be the quaternary ammonium fluorides with detergent properties. Certain cationic surfactants may also act as germicides in the oral care compositions.

Nonionic surfactants may be broadly defined as compounds produced by the condensation of alkylene oxide groups (hydrophilic in nature) with an organic hydrophobic compound which may be aliphatic or alkylaromatic in nature. Examples of suitable nonionic surfactants include the pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Zwitterionic synthetic surfactants may be broadly described as derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

Betaine surfactants include decyl betaine or 2-(N-decyl-N,N-dimethylammonio)acetate, coco betaine or 2-(N-coco-N,N-dimethyl ammonio)acetate, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, lauramidopropyl betaine and the like. In some embodiments, the betaines are cocoamidopropyl betaine or lauramidopropyl betaine.

Surfactants may be present at a concentration in the range of about 0.1% to about 2.5%, from about 0.3% to about 2.5%, or from about 0.5% to about 2.0% by weight of the total composition.

3. Anti-plaque Agent

The oral care composition may also include an anti-plaque agent, such as a synthetic anionic polymer, e.g., polyacrylate or copolymers of maleic anhydride or acid and methyl vinyl ether as well as polyamino propane sulfonic acid (AMPS), zinc citrate trihydrate, polypeptides (such as polyaspartic and polyglutamic acids), and mixtures thereof.

4. Chelating Agents

The oral care compositions may include a chelating agent. Chelating agents include tartaric acid and pharmaceutically-acceptable salts thereof, citric acid and alkali metal citrates and mixtures thereof. Chelating agents may complex calcium found in the cell walls of bacteria. Chelating agents may also disrupt plaque by removing calcium from the calcium bridges which help hold this biomass intact. A chelating agent that may result in tooth demineralization may not be used.

In particular embodiments, sodium and potassium citrate are alkali metal citrates that may be in the subject composition. In certain cases, chelating agents may include a citric acid/alkali metal citrate combination. In other cases, alkali metal salts of tartaric acid may be used. Other agents include disodium tartrate, dipotassium tartrate, sodium potassium tartrate, sodium hydrogen tartrate and potassium hydrogen tartrate.

The amounts of chelating agent may be about 0.1% to about 2.5%, about 0.5% to about 2.5%, or from about 1.0% to about 2.5%. The tartaric acid salt chelating agent may be used alone or in combination with other optional chelating agents. In some embodiments, these chelating agents may have a calcium binding constant of about 10¹ to 10⁵ to provide improved cleaning with reduced plaque formation.

Another group of chelating agents is the anionic polymeric polycarboxylates. Such materials are well known in the art, being employed in the form of their free acids, partially or fully neutralized water soluble alkali metal (e.g. potassium and preferably sodium) or ammonium salts. In certain cases, composition may contain 1:4 to 4:1 copolymers of maleic anhydride or acid with another polymerizable ethylenically unsaturated monomer, such as methyl vinyl ether (methoxyethylene) having an average molecular weight (AMW) of about 30,000 to about 1,000,000.

Other operative polymeric polycarboxylates include those such as the 1:1 copolymers of maleic anhydride with ethyl acrylate, hydroxyethyl methacrylate, N-vinyl-2-pyrrolidone, or ethylene, and 1:1 copolymers of acrylic acid with methyl or hydroxyethyl methacrylate, methyl or ethyl acrylate, isobutyl vinyl ether or N-vinyl-2-pyrrolidone.

Additional operative polymeric polycarboxylates may be copolymers of maleic anhydride with styrene, isobutylene or ethyl vinyl ether, polyacrylic, polyitaconic and polymaleic acids, and sulfoacrylic oligomers of AMW as low as 1,000.

5. Fluoride Source

In some embodiments, a water-soluble fluoride compound may be added to the oral care composition in an amount sufficient to give a fluoride ion concentration in the composition at 25° C., from about 0.0025% to about 5.0% by weight, or about 0.005% to about 2.0% by weight. A wide variety of fluoride ion-yielding materials may be employed as sources of soluble fluoride in the present compositions. Representative fluoride ion sources may include stannous fluoride, sodium fluoride, potassium fluoride, sodium monofluorophosphate and many others. In certain cases, the subject composition may contain stannous fluoride and sodium fluoride, as well as mixtures thereof.

6. Teeth Whitening Actives and Teeth Color Modifying Substances

A teeth whitening agent and/or teeth color modifying substance may also be present in the oral care composition. These substances are suitable for modifying the color of the teeth. These substances may comprise particles that when applied on the tooth surface modify that surface in terms of absorption and, or reflection of light. Such particles may provide an appearance benefit when a film containing such particles is applied over the surfaces of a tooth or teeth.

Particles in the oral care compositions include pigments and colorants routinely used in the cosmetic arts. There are no specific limitations as to the pigment and, or colorant used in the present composition. Pigments and colorants may include inorganic white pigments, inorganic colored pigments, pearling agents, filler powders and the like. Specific examples may be selected from the group consisting of talc, mica, magnesium carbonate, calcium carbonate, magnesium silicate, aluminum magnesium silicate, silica, titanium dioxide, zinc oxide, red iron oxide, brown iron oxide, yellow iron oxide, black iron oxide, ferric ammonium ferrocyanide, manganese violet, ultramarine, nylon powder, polyethylene powder, methacrylate powder, polystyrene powder, silk powder, crystalline cellulose, starch, titanated mica, iron oxide titanated mica, bismuth oxychloride, and mixtures thereof. In certain embodiments, the subject composition contains those selected from the group consisting of titanium dioxide, bismuth oxychloride, zinc oxide and mixtures thereof.

The pigments may be used as opacifiers and colorants. These pigments may be used as treated particles, or as the raw pigments themselves. Typical pigment levels may be selected for the particular impact that is desired by the consumer. For example, for teeth that are particularly dark or stained one may use pigments in sufficient amount to lighten the teeth. On the other hand, where individual teeth or spots on the teeth are lighter than other teeth, pigments to darken the teeth may be useful. The levels of pigments and colorants may be used in the range of about 0.05% to about 20%, from about 0.10% to about 15%, or from about 0.25% to about 10% of the composition.

7. Thickening Agents

In some embodiments such as toothpaste or gels, thickening material may be added to provide a consistency to the composition, active release characteristics upon use, shelf and other stability, and the like. Thickening agents include carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth may also be used. Colloidal magnesium aluminum silicate or finely divided silica may be used as part of the thickening agent to further improve texture.

Thickening or gelling agents may include a class of homopolymers of acrylic acid crosslinked with an alkyl ether of pentaerythritol or an alkyl ether of sucrose, or carbomers, or mixtures thereof.

Copolymers of lactide and glycolide monomers having the molecular weight in the range of from about 1,000 to about 120,000 (number average), may be used in the subject composition such as a “subgingival gel.”

Thickening agents may be present in an amount of from about 0.1% to about 15%, about 2% to about 10%, or from about 4% to about 8%, by weight of a total toothpaste or gel composition. Higher concentrations may be used for chewing gums, lozenges (including breath mints), sachets, non-abrasive gels and subgingival gels.

8. Humectants

In some embodiments, the oral care composition includes a humectant to prevent the composition from hardening upon exposure to air, to give the composition a moist feel to the mouth, and, in the case of particular humectants, to impart desirable sweetness of flavor to, e.g., a toothpaste composition. The humectant, on a pure humectant basis, may comprise from about 0% to about 70%, or about 5% to about 25%, by weight of the compositions herein. Suitable humectants for use in compositions of the subject invention include edible polyhydric alcohols such as glycerin, sorbitol, xylitol, butylene glycol, polyethylene glycol, and propylene glycol. In certain cases, the subject composition contains humectants such as sorbitol and glycerin.

9. Flavoring and Sweetening Agents

Flavoring agents may also be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof. Flavoring agents may be present at levels of from about 0.001% to about 5%, by weight of the composition.

Suitable sweetening agents include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, cyclamate salts, sodium cyclamate or sodium saccharin, and mixtures thereof. These agents may be present at from about 0.1% to about 10%, or from about 0.1% to about 1%, by weight, of the total composition.

In addition to flavoring and sweetening agents, coolants, salivating agents, warming agents, and numbing agents may be used as optional ingredients in the subject composition. These agents may be present at a level of from about 0.001% to about 10%, or from about 0.1% to about 1%, by weight of the composition.

The coolant may be any of a wide variety of materials, including carboxamides, menthol, ketals, diols, and mixtures thereof. Coolants include paramenthan carboxyamide agents such as N-ethyl-p-menthan-3-carboxamide, N,2,3-trimethyl-2-isopropylbutanamide, and mixtures thereof. Additional coolants may be selected from the group consisting of menthol, 3-1-menthoxypropane-1,2-diol, menthone glycerol acetal, and menthyl lactate. The terms menthol and menthyl include dextro- and levorotatory isomers of these compounds and racemic mixtures thereof.

Warming agents include capsicum and nicotinate esters, such as benzyl nicotinate. Numbing agents include benzocaine, lidocaine, clove bud oil, and ethanol.

10. Alkali Metal Bicarbonate Salt

The oral care composition may also include an alkali metal bicarbonate salt Alkali metal bicarbonate salts may be soluble in water and, unless stabilized, may release carbon dioxide in an aqueous system. Sodium bicarbonate, also known as baking soda, may be present as the alkali metal bicarbonate salt in the subject composition. In certain embodiments, the subject composition may contain from about 0.5% to about 30%, about 0.5% to about 15%, or from about 0.5% to about 5% of an alkali metal bicarbonate salt.

11. Miscellaneous Carriers

Water employed in the preparation of the oral care compositions may be of low ion content and free of organic impurities. Water may be from about 5% to about 70%, or from about 20% to about 50%, by weight of the aqueous compositions. These amounts of water include free water that is added directly as well as water that is introduced as part of other agents or carriers.

Poloxamers may be employed in the oral care compositions. A poloxamer may be classified as a nonionic surfactant. It may also function as an emulsifying agent, binder, stabilizer, and other related functions. Poloxamers may be difunctional block-polymers terminating in primary hydroxyl groups with molecular weights ranging from 1,000 to above 15,000.

Other emulsifying agents that may be used in the subject compositions include polymeric emulsifiers. Predominantly high molecular weight polyacrylic acid polymers may be useful as emulsifiers.

Titanium dioxide may also be added to the oral care composition. Titanium dioxide is a white powder which may add opacity to the compositions. Titanium dioxide may be present in an amount of from about 0.25% to about 5% by weight of the subject composition.

The pH of the subject composition is preferably preselected based on the use of one or more buffering agents. The buffering agents may be suitable for maintaining the pH of a composition in the range of about pH 4.0 to about pH 10.0. Buffering agents include monosodium phosphate, trisodium phosphate, sodium hydroxide, sodium carbonate, sodium acid pyrophosphate, citric acid, and sodium citrate. Buffering agents may be administered at a level of from about 0.5% to about 10%, by weight of the oral care compositions. In certain embodiments, the pH of dentifrice compositions may be measured from a 3:1 aqueous slurry of dentifrice, e.g., 3 parts water to 1 part dentifrice.

Other agents that may be used in the present compositions include dimethicone copolyols selected from alkyl- and alkoxy-dimethicone copolyols, such as C12 to C20 alkyl dimethicone copolyols and mixtures thereof. In certain cases, the subject composition contain cetyl dimethicone copolyol. The dimethicone copolyol may be present in a level of from about 0.01% to about 25%, about 0.1% to about 5%, or from about 0.5% to about 1.5% by weight. The dimethicone copolyols may aid in providing positive tooth feel benefits.

12. Other Active Agents

The subject oral care composition may also include other active agents, such as antimicrobial agents. Included among such are water insoluble non-cationic antimicrobial agents such as halogenated diphenyl ethers, phenolic compounds including phenol and its homologs, mono and poly-alkyl and aromatic halophenols, resorcinol and its derivatives, bisphenolic compounds and halogenated salicylanilides, benzoic esters, and halogenated carbanilides. The water soluble antimicrobials may include quaternary ammonium salts and bis-biquamide salts, among others. Triclosan monophosphate is an additional water soluble antimicrobial agent. The quaternary ammonium agents may include those in which one or two of the substitutes on the quaternary nitrogen has a carbon chain length (typically alkyl group) from about 8 to about 20, or from about 10 to about 18 carbon atoms while the remaining substitutes (typically alkyl or benzyl group) have a lower number of carbon atoms, such as from about 1 to about 7 carbon atoms, such as methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl)ammonium bromide, benzyl dimethylstearyl ammonium chloride, cetyl pyridinium chloride, quaternized 5-amino-1,3-bis(2-ethyl-hexyl)-5-methyl-hexahydropyrimidine, benzalkonium chloride, benzethonium chloride and methyl benzethonium chloride are exemplary of quaternary ammonium antibacterial agents. Other compounds may be bis[4-(R-amino)-1-pyridinium]alkanes. Other antimicrobials such as copper bisglycinate, copper glycinate, zinc citrate, and zinc lactate may also be included.

In addition to a subject mutanase, a subject oral care composition may also contain one or more other enzymes that have carbohydrate hydrolysis, antimicrobial or teeth whitening activities. Such enzymes include, but are not limited to, a deaminase, an esterase, a glycosidase, a lipase, an oxidase, a peroxidase, a protease, a urease and a cellulase.

In certain cases, the composition may contain enzymes other than an oxidase, e.g., a laccase, peroxidas, dextranase, or a mutanase. These enzymes may have antimicrobial activity, a bleaching activity, or both. Other useful enzymes include proteases, lytic enzymes, and plaque matrix inhibitors. Proteases include papain, pepsin, trypsin, ficin, bromelin; cell wall lytic enzymes include lysozyme; plaque matrix inhibitors include dextranases, and mutanases. The effect of certain enzymes may be obtained by direct oxidation of the teeth stains or via a mediator.

### III. Methods of Use

Further provided is a method involving contacting an enamel surface of a tooth with a composition as described under conditions suitable for binding of the oxidase present in the composition to the enamel surface and production of peroxide, either during the contacting step, or after the contacting step. Relatedly, a method of binding an oxidase to a tooth surface is provided, which method involves contacting a tooth having an enamel surface with an oxidase in the presence of polyethyleneimine or sorbitol under conditions that provide for binding of the oxidase to the enamel surface via the polyethyleneimine or the sorbitol, and the production of peroxide. The enzyme may be bound directly or indirectly to the enamel surface of the tooth via the PEI or sorbitol.

In some embodiments, the methods result in cleaning, whitening, and/or polishing teeth, reducing the incidence of stain, plaque, gingivitis and calculus on dental enamel, and combinations thereof.

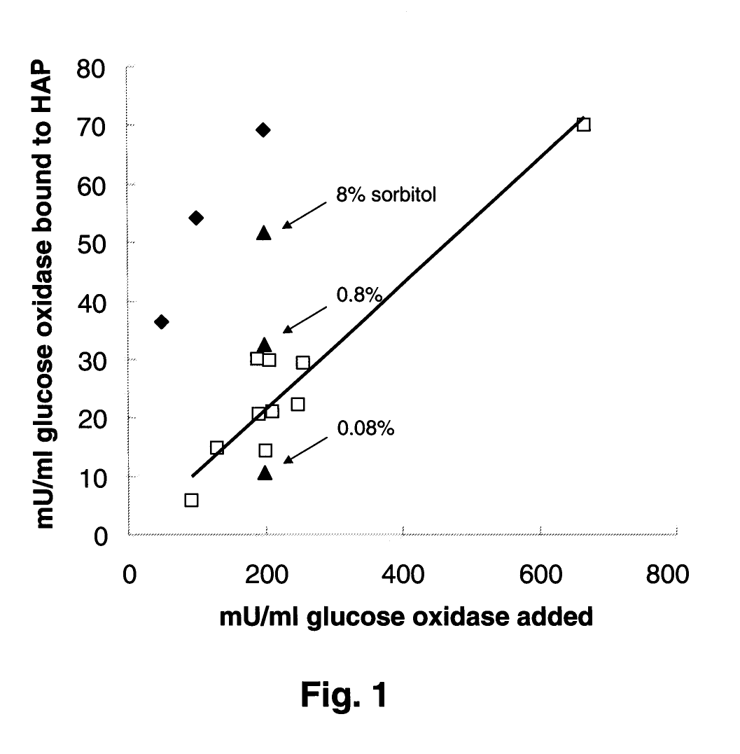

In some embodiments, the method effectively deposits oxidase on the contacted enamel surface. The deposition may be from aqueous-based formulations such as cleaning and detergent compositions, or from essentially non-aqueous based formulations, which comprise polyethyleneimine or sorbitol. As indicated in FIG. 1, when applied to a suitable surface, an exemplary oral composition comprising polyethyleneimine increases the retention of glucose oxidase on a hard surface.

The present methods may be performed, e.g., by brushing, rinsing, chewing, or otherwise introducing the described compositions into the oral cavity for contacting a tooth surface. Methods of contacting include the use of a topical oral gel, mouthspray, or other form of the oral composition, such as strips, films, or floss, which contact the teeth and/or oral mucosa. The composition may be applied directly to the teeth, gums, or other oral surface with a brush, a pen applicator, a doe's foot applicator, a finger, or the like. The method may be performed entirely or partially by a consumer/subject/end-user, e.g., in a domestic setting, or by a dental health professional, e.g., in a clinical setting

### EXPERIMENTAL

The following example is provided to demonstrate and further illustrate how adherence of glucose oxidase to hydroxyapatite increases in the presence of either polyethyleneimine or sorbitol.

### Example 1

### Glucose Oxidase Binding to Saliva-Coated Hydroxyapatite Disks

Hydroxyapatite (HAP) disks coated with saliva are commonly used as models of a tooth surface. This example demonstrates that the addition of either sorbitol or polyethyleneimine improves the binding of glucose oxidase in a catalytically active conformation to saliva-coated hydroxyapatite.

Saliva was collected from 3-5 donors, clarified by centrifugation, and added to cover HAP disks (5 mm×2.0 mm Clarkson Chromatography) in a covered plastic tray. The disks were incubated with the saliva overnight, with gentle shaking, at room temperature. The disks were rinsed twice with 50 mM phosphate buffer prior to use in the binding assay. Varying concentrations of glucose oxidase (50-650 mU/ml) were added to the saliva-coated HAP disks in the presence of sorbitol (0.8-8%) or polyethyleneimine (1%). The amount of glucose oxidase retained on the hydroxyapatite disks, following three consecutive washes with phosphate buffered saline, was measured using a fluorescence (Amplex Red, Molecular Probes #A22189) horseradish peroxidase coupled assay, as per manufacturer's recommendation.

As is clearly seen in the experimental result, the addition of at least 0.8% sorbitol or 1% polyethyleneimine increased the amount of active glucose oxidase detected on the hydroxyapatite.

1. An oral care composition comprising
a) an orally acceptable carrier;
b) polyethyleneimine; and
c) an uncomplexed oxidase.

2. The oral care composition of claim 1, wherein said oxidase is glucose oxidase.

3. The composition of claim 1, wherein said polyethyleneimine and said uncomplexed glucose oxidase are present in amounts sufficient for binding of said oxidase to an enamel surface of a tooth and production of peroxide in the presence of a suitable substrate for said oxidase.

4. The oral care composition of claim 1, wherein said polyethyleneimine is present in said oral care composition at a concentration in the range of 0.001% to 10% by weight of said composition.

5. The oral care composition of claim 1, wherein said oxidase is present in said oral care composition at a concentration in the range of 0.0001% to 1% by weight of said composition.

6. The oral care composition of claim 1, wherein said oral care composition comprises at least one of a thickener, a surfactant, a humectant, and an abrasive.

7. The oral care composition of claim 1, wherein said oral care composition comprises a second enzyme in addition to said oxidase.

8. The oral care composition of claim 7, wherein said oxidase is not complexed with another enzyme.

9. The oral care composition of claim 1, wherein said oral care composition is fluid.

10. The oral care composition of claim 1, wherein said oral care composition is solid.

11. The oral care composition of claim 9, wherein said oral care composition is formulated as a toothpaste.

12. The oral care composition of claim 10, wherein said oral care composition is formulated as a tooth floss.

13. A method of producing peroxide on a tooth surface comprising:
contacting a composition of claim 1 with an enamel surface of a tooth under conditions suitable for binding of said oxidase to said enamel surface and production of peroxide.

14. The method of claim 13, wherein contacting is performed using a toothbrush.

15. The method of claim 13, wherein contacting is performed using tooth floss.

16. The method of claim 13, wherein said method results in death of bacteria on said tooth and/or tooth whitening.

17. A method of binding oxidase to a tooth comprising:
contacting a tooth having an enamel surface with an oxidase in the presence of polyethyleneimine or sorbitol under conditions that provide for binding of said oxidase to said enamel surface via said polyethyleneimine or said sorbitol and production of peroxide.

18. The method of claim 17, wherein said contacting is performed using a toothbrush.

19. The method of claim 17, wherein said contacting is performed using tooth floss.
